(19) United States
(12) Patent Application Publication
(10) Pub. No.: US 2024/0423959 A1
(43) Pub. Date: Dec. 26, 2024

(54) TOPICAL BENZIMIDAZOLE FORMULATIONS AND METHODS FOR USE IN TREATING INFLAMMATORY DERMATOSES

(71) Applicant: JJR&D, LLC, Celina, TN (US)

(72) Inventors: Joaquin J. Jimenez, Miami, FL (US); Naiem T. Issa, Miami, FL (US); John P. McCook, Frisco, TX (US)

(21) Appl. No.: 18/212,065

(22) Filed: Jun. 20, 2023

Related U.S. Application Data
(60) Provisional application No. 63/353,907, filed on Jun. 21, 2022.

(51) Int. Cl. A61K 31/4184; A61K 9/06; A61K 47/10; A61K 47/14; A61K 47/22; A61K 47/38; A61K 47/40; A61K 47/44; A61P 17/00; A61P 29/00

(52) U.S. Cl. CPC A61K 31/4184; A61K 9/06; A61K 47/10; A61K 47/14; A61K 47/22; A61K 47/38; A61K 47/40; A61K 47/44; A61P 17/00; A61P 29/00

(57) ABSTRACT

Compositions and methods for treating and preventing inflammatory and autoimmune skin conditions, particularly rosacea, using one or more topically applied benzimidazole compounds in a pharmaceutically acceptable carrier for use on skin. A preferred benzimidazole compound comprises mebendazole. A treatment composition preferably comprises 0.05-0.20 weight percent mebendazole, and may comprise up to 20.0% mebendazole, in an aqueous or non-aqueous carrier or vehicle comprising a cream, gel, lotion, liquid, emulsion, microemulsion, aerosol spray, non-aerosol spray, serum, solution, suspension, or ointment and is applied at least once daily over a treatment period of at least two weeks to result in a reduction of cutaneous cytotoxic CD+8 T-cells, papules, pustules, swelling, appearance of redness or inflammation, and/or itchiness in the affected area compared to pre-treatment levels.

### CROSS REFERENCE TO RELATED APPLICATION

This application claims the benefit of U.S. Provisional Application Ser. No. 63/353,907 filed on Jun. 21, 2023.

### BACKGROUND OF THE INVENTION

### 1. Field of the Invention

The present invention relates to topical formulations of benzimidazoles, and specifically of mebendazole, and their use in treating and preventing inflammatory and other skin diseases and conditions, such as rosacea.

### 2. Description of Related Technology

The use of benzimidazoles, including mebendazole, for treatment of various health related conditions in humans and non-human animals is known in the art. Mebendazole is an approved anthelminthic drug with favorable toxicity profile. It is generally given orally for intestinal helminthiasis. Dosing regimens have ranged from short-term low-dose treatments to long-term high-dose treatments over several months. For example, U.S. Pat. Nos. 9,877,950 and 5,169,846 disclose the use of mebendazole as an anthelminthic for treatment of parasites and worms in animals.

Interestingly, mebendazole has also been shown to have significant antiproliferative activity across various cancer models. These include glioblastoma, breast, lung, ovarian, colon, osteosarcoma, melanoma cell lines among others. Clinical trials are currently assessing the utility of mebendazole for high-grade glioma, medulloblastoma and metastatic gastrointestinal cancer. For example, WO2002058697 discloses the use of mebendazole for the treatment of cancers, including through topical application. Similarly, US20210369679 discloses the use of mebendazole with a non-steroidal anti-inflammatory (NSAID) for treating cancer, including skin cancer.

The anti-helminthic effect of mebendazole is attributed to its inhibition of tubulin, which disrupts the organism's cytoskeletal network leading to parasite death. Mebendazole also inhibits mammalian tubulin but with lesser binding affinity. It is believed that inhibition of tubulin is also one mechanism by which mebendazole exerts a cytotoxic effect on tumor cells. In addition to its ability to bind and inhibit tubulin polymerization, mebendazole also binds and inhibits numerous cytoplasmic kinases that are critical to cancer initiation, progression and metastasis. Examples of these kinases include ABL1, BRAF, DYRK1B, JAK3, and PDGFR.

The immunomodulatory effects of mebendazole have been cursorily studied in in vitro models. For example, mebendazole has been found to cause an M2 to M1 phenotypic switch in monocyte/macrophage models. This could potentially explain the benefit of mebendazole in solid malignancies beyond its direct antiproliferative effect on tumor cells as the M1 macrophages induce direct and indirect anti-tumor effects. Furthermore, mebendazole has been shown to uniquely upregulate ERK signaling in THP-1 monocytoid cells and human CD4+ T-cells isolated from patients with systemic lupus erythematosus with known defective ERK signaling, an effect that is unique to mebendazole and not the other benzimidazoles. It is therefore postulated that mebendazole may be of use in autoimmune diseases characterized by defective ERK signaling. Mebendazole treatment of scleroderma is disclosed, for example, in WO2019112031. It has not, however, been known to use benzimidazoles for the topical treatment of other inflammatory or autoimmune diseases and conditions of the skin, such as rosacea.

Rosacea is one of the most common inflammatory skin disorders in humans. Rosacea is a chronic inflammatory skin disorder characterized by facial flushing, telangiectasias, irritation, pain, papules, pustules and phymatous/granulomatous changes. There are four major subtypes: erythematotelangiectactic rosacea (ETR), papulopustular rosacea (PPR), phymatous rosacea (PR), and ocular rosacea (OR). While initially thought to be vascular-driven disorder, rosacea is now appreciated to have a major immunologic component driving the disease process. This immunologic component is driven by numerous processes: pro-inflammatory neuropeptide release by cutaneous nerve endings, changes in the inflammatory milieu by the local microbiome, components of resident innate immunity altering the cytokine milieu, among others. With respect to adaptive immunity, presence and proliferation of CD4+ and CD8+ T-cells have been shown to be increased in ETR and PPR compared to healthy skin.

It is postulated that reduction of cutaneous inflammation leads to improvement of rosacea. This is indeed demonstrated by the use of topical and systemic anti-microbials, corticosteroids and other non-steroidal immunomodulators. Examples of such treatments include but are not limited to ivermectin, metronidazole, tetracyclines, prednisone, among others. As CD8+ T-cells are up-regulated in rosacea, it is also postulated that reducing/preventing the activity, proliferation or viability of CD8+ T-cells is a viable treatment strategy.

While the immunomodulatory effects of mebendazole have been investigated in monocytes/macrophages and CD4+ T-cells, its effect on CD8+ T-cells has not previously been investigated. There is a need in the art for an improved immunomodulatory treatment for inflammatory or autoimmune diseases and conditions of the skin in humans, and particularly treatment of rosacea. According to preferred embodiments of the invention, use of mebendazole results in such improvements, particularly in immunomodulatory effects on CD8+ T-cells.

There is also a need in the art for a mebendazole treatment composition that may be used for treating such skin conditions that has improved solubility and skin penetration ability. Mebendazole is poorly soluble in water with an aqueous solubility of approximately 0.035 mg/mL for mebendazole Polymorph C at 25° C. In addition to negligible solubility in water, mebendazole has negligible solubility in most solvents other than DMSO (approximately 2% by weight). Mebendazole is also freely soluble in formic acid. However, DMSO and formic acid are not suitable as pharmaceutical vehicle ingredients for a skin treatment product, particularly a facial skin treatment composition (as rosacea primarily impacts the face), due to safety considerations. Thus, there is a need for a process and composition ingredients to increase the solubility of mebendazole in a pharmaceutical vehicle that would be appropriate for use on human and animal skin, particularly human facial skin.

### SUMMARY OF THE INVENTION

According to one preferred embodiment of the invention, a treatment composition comprising one or more benzimidazoles in a pharmaceutically acceptable vehicle for topical application is used for the treatment of an inflammatory or autoimmune disease and/or condition of the skin. According to a preferred embodiment, the disease or condition treated with a composition according to the invention is one affecting only animals (non-humans), only humans, or both animals and humans. Most preferably, compositions according to preferred embodiments are used for the treatment of rosacea in humans.

According to one preferred embodiment, the treatment composition comprises mebendazole or a salt of mebendazole or methyl 5-benzoylbenzimidazole-2-carbamate (each referred to herein generally as MBZ). In other embodiments, the treatment composition comprises one or more of albenzadole, thiabenzadole, fenbenzadole, MBZ, or salts of the foregoing. Except as referenced in the claims, any other benzimidazoles may be substituted for or used in addition to mebendazole or MBZ referenced herein in connection with the description of the invention and preferred embodiments of the invention. Mebenzadole exists in several polymorph crystalline states that include Polymorph A, B, and C. Polymorph C is the preferred form for compositions of this invention, but the others may also be used or may be excluded. Salts of mebenzadole may include mebendazolium lactate, mebendazolium glycolate, and mebendazolium mesylate.

According to preferred embodiments, an MBZ treatment composition comprises around 0.01-1.0% mebendazole by weight, more preferably around 0.05-0.25% by weight, and most preferably around 0.05-0.15% by weight, in an MBZ gel composition, an MBZ cream base vehicle, or in another pharmaceutically acceptable vehicle for topical use with human and/or animal skin, with or without other active ingredients. Such amounts of MBZ are particularly preferred when the vehicle for the MBZ treatment composition is a clear aqueous or anhydrous solution or gel. According to another preferred embodiment, the composition comprises (1) around 0.01-0.50% mebendazole by weight, more preferably around 0.05-0.25%, and most preferably around 0.05-0.15%; (2) around 10-60% by weight total of one or more solvents, penetrants, and/or emulsifying ingredients, more preferably around 25-60%, most preferably around 40-55%; and (3) water. One or more other active ingredients may also be included in these embodiments. In these embodiments where lower amounts of MBZ are used, the solvents and/or other ingredients preferably increase the solubility of MBZ to at least 5 times, more preferably at least 10 times or more of the solubility of MBZ in water, allowing less MBZ to be used to achieve preferred skin penetration performance.

According to other preferred embodiments, an MBZ treatment composition comprises a vehicle that does not increase the solubility of MBZ as much as other preferred embodiments, thus requiring more MBZ to achieve to increase the skin penetration performance of such MBZ treatment compositions. One such vehicle is Vanicream® as described herein. In these embodiments, an MBZ treatment composition comprises 1.0-20.0% mebendazole by weight, more preferably around 1.0-10.0%, and most preferably around 1.0-5.0%. Such amounts of MBZ are particularly preferred when the vehicle for the MBZ treatment composition is an aqueous lotion, cream, emulsion or anhydrous suspension of mebendazole wherein the mebendozole is mostly in a non-soluble, crystalline form. In these embodiments where higher amounts of MBZ are used, the solvents and/or other ingredients preferably increase the solubility of MBZ to no more than 10 times, more preferably no more than 5 times or less of the solubility of MBZ in water.

According to certain preferred embodiments, the mebendazole (or other benzimidazole) is physically nanosized in the composition. According to other preferred embodiments, the mebendazole (or other benzimidazole) is also preferably suspended and/or non-solubilized mebendazole and is encapsulated in the composition. According to still other preferred embodiments, the mebendazole (or other benzimidazole) is solubilized as much as possible with non-DMSO solvents and/or surfactants. According to other preferred embodiments, the mebendazole (or other benzimidazole) and at least one solvent are processed through a microfluidizer to form an MBZ (or other benzimidazole) concentrate composition and/or an MBZ (or other benzimidazole) treatment composition. According to another preferred embodiment, the mebendazole (or other benzimidazole) and at least one solvent are not processed through a microfluidizer to form an MBZ (or other benzimidazole) concentrate composition and/or an MBZ (or other benzimidazole) treatment composition. These variations on the mebendazole (or other benzimidazole) may be used with any compositions comprising mebendazole (or other benzimidazole) according to preferred embodiments of the compositions herein.

According to another preferred embodiment, the vehicle in the composition comprises an aqueous lotion, cream, gel, solution, suspension, or ointment. According to still another preferred embodiment, the vehicle in the composition comprises a non-aqueous lotion, cream, solution, suspension, gel or ointment. Preferred embodiments for a vehicle comprise one or more of the following, preferably all of the following: (1) water (preferably purified or deionized), (2) petrolatum, (3) sorbitol, (4) cetearyl alcohol, (5) propylene glycol, (6) ceteareth-20 (a polyethylene glycol ether of cetearyl alcohol), (7) simethicone, (8) glyceryl stearate, (9) PEG-30 Stearate (a polyethylene glycol ester of stearic acid), (10) sorbic acid, and (11) BHT (butylated hydroxytoluene). Other preferred embodiments for a vehicle comprise one or more of the following, preferably all of the following: (1) water (preferably purified or deionized), (2) glycerin, (3) polyacrylic acid (preferably carbomer), (4) disodium ethylenediaminetetraacetic acid (EDTA), (5) cetyl alcohol, (6) stearyl alcohol, (7) glyceryl stearate and/or PEG 100 stearate, (8) polysorbate 80, (9) triethanolamine, and (10) phenoxyethanol. Any ingredient listed as potentially included in one preferred embodiment, may also be excluded in another preferred embodiment.

According to one preferred embodiment, an MBZ concentrate composition comprises: (1) mebendazole (or other benzimidazole) and (2) one or more compounds or ingredients that act as solvents, skin penetrants, and/or emulsifying agents. According to another preferred embodiment, there are at least two, more preferably at least three, ingredients in category (2). Preferably, at least one ingredients in category (2) acts as a solvent and an emulsifying agent. According to another preferred embodiment, an MBZ concentrate composition comprises: (1) mebendazole (or other benzimidazole); (2) a sorbitol based solvent, (3) an ethylene and/or propylene glycol based solvent, and (4) a solubilizer and/or emulsifying agent. These preferred MBZ concentrate compositions comprise around 1.0-10.0% by weight mebendazole, more preferably around 3.0-6.0%, most preferably around 3.0-4.0%. According to still other preferred embodiments, the amount of mebendazole in an MBZ concentrate composition is reduced to around 0.05-1.0% by weight, more preferably 0.05-0.2%, and most preferably 0.05-0.15%.

A preferred ethylene glycol based solvent is diethylene glycol monoethyl ether (Transcutol P®), but others may also be used. A preferred propylene glycol based solvent is propylene glycol monolaurate (Lauryl Glycol®), but others may also be used. According to another preferred embodiment, an MBZ concentrate composition comprises around 15.00-25.00% by weight total of the ethylene and/or propylene glycol based solvent, more preferably around 18.00-21.00%. In some preferred embodiments, both an ethylene glycol based solvent and a propylene glycol based solvent are used together in an MBZ concentrate composition in weight ratios of around 80:20 to 70:30, more preferably around 75:25 to 65:35, and most preferably around 60:40.

A preferred sorbitol based solvent is dimethyl isosorbide (DMI). According to another preferred embodiment, an MBZ concentrate composition comprises around 32.00-45.00% by weight of the sorbitol based solvent, more preferably around 36.00-40.00%.

A preferred solubilizer and/or emulsifying agent is polyoxyl 40 hydrogenated castor oil (Cremophor RH 40). According to another preferred embodiment, an MBZ concentrate composition comprises around 32.00-45.00% by weight of the solubilizer and/or emulsifying agent, more preferably around 36.00-40.00%.

The solvents used in compositions according to preferred embodiments are (1) preferably capable of dissolving MBZ at 10× or more the solubility of MBZ in water (which is approximately 35 μg/mL) and (2) suitable for use, in amounts necessary to dissolve MBZ according to preferred embodiments, on human or animal skin. According to still other preferred embodiments, other solvents/skin penetrants that may be used in addition to or as alternates to dimethyl isosorbide, diethylene glycol monoethyl ether, and Cremophor RH 40 include: caprylocaproyl macrogol-8 glycerides, propylene glycol monocaprylate. polyglyceryl-3 dioleate; diisopropyl adipate, diethylhexyl adipate, poly(glyceryl) adipate.

According to one preferred embodiment, an MBZ concentrate composition according to preferred embodiments herein is made by: (1) mixing the one or more solvents and mebendazole to form a first mixture; (2) heating the first mixture to a temperature within a first temperature range for a first period of time; and (3) mixing or stirring during the heating step, such as by using a magnetic stirrer. According to one preferred embodiment, the first period of time is around 5 to 20 minutes, more preferably around 5 to 15 minutes. Most preferably, the heating temperature and first period of time will not result in degradation of the MBZ of more than 10%, more preferably not more than 8% and most preferably not more than 5%. The first temperature range is most preferably between at least 60° C. but less than a temperature at which MBZ will experience degradation of 5% or more. More preferably, the first temperature range is 60-90° C., and most preferably 70-90° C. Preferably, steps 1-3 are carried out before mixing the MBZ or the first mixture with any water or aqueous based application vehicle.

According to one preferred embodiment, an MBZ gel composition comprises (1) mebendazole (or other benzimidazole); (2) water, and (3) a gelling or thickening agent. According to another preferred embodiment, an MBZ gel composition comprises (1) an MBZ concentrate composition, (2) water, and (3) a gelling or thickening agent. According to other preferred embodiments, an MBZ gel composition further comprises (4) a cyclodextrin compound to aid in solubility/reduce precipitation of the MBZ. A preferred cyclodextrin compound is hydroxypropyl beta cyclodextrin (Cavasol® W7 HP). Other cyclodextrin compounds comprising alpha- and gamma-cyclodextrins and alternate chemical modifications of the alpha, beta and gamma cyclodextrins in addition to hydroxyalkylation including, methylation, ethylation, phosphated and sulfonated, may also be used.

According to other preferred embodiments, an MBZ treatment composition comprises an MBZ gel composition that may be mixed with a vehicle or applied to skin for treatment without mixing with additional vehicle ingredients. According to other preferred embodiments, an MBZ treatment composition comprises an MBZ concentrate composition that is mixed with a vehicle prior to application to skin for treatment.

According to another preferred embodiment, a method of treating an inflammatory or autoimmune disease and condition of the skin comprises topically applying a composition comprising one or more benzimidazoles in a pharmaceutically acceptable vehicle to an affected area of the skin at least once daily for a treatment period of at least two weeks, more preferably at least twelve weeks and continue to apply the composition to the affected facial area daily or every other day to prevent reoccurrence of a new rosacea outbreak. Most preferably, the composition used in the preferred methods is one according to a preferred embodiment of the invention. According to one preferred method, a composition comprising mebendazole is applied to the affected area of the skin at a dosage rate of around 100 to 500 milligrams, more preferably 50 to 250 milligrams to each side of the face to the affected areas.

Use of treatment compositions and methods according to preferred embodiments of the invention have been shown to be effective in reducing viability of T-cells and in treating a patient with rosacea. The inventors have found that use of mebendazole according to a preferred embodiment reduced the viability of T-cells in an in vitro model. Use of preferred methods and composition ingredients for making an MBZ concentrate composition, an MBZ gel composition, and/or an MBZ treatment composition have also been shown to result in increased skin penetration and/or increased solubility of MBZ in a pharmaceutical vehicle appropriate for use on human and animal skin, particularly human facial skin. Accordingly, the methods and compositions of the preferred embodiments provide advantages as a therapeutic agent for immunomodulation, particularly through a suppressive effect on CD8+ T-cells, for treatment of inflammatory and autoimmune diseases and conditions of the skin, particularly rosacea, with improved penetration and solubility of the MBZ.

### BRIEF DESCRIPTION OF THE DRAWINGS

The compositions and methods of the invention are further described and explained in relation to the following figures wherein:

FIG. 1 is a graph depicting the reduction in density of T-lymphocytes when exposed to various concentrations of a composition comprising mebendazole;

FIG. 2 is a graph depicting a reduction in the number of papules and pustules on the left (treated) cheek of a patient with rosacea when treated with a cream composition comprising 10% mebendazole by weight;

FIG. 3 contains photographs showing the left (treated) and right (untreated/control) cheeks of the patient with rosacea referenced in FIG. 2.

### DETAILED DESCRIPTION OF PREFERRED EMBODIMENTS

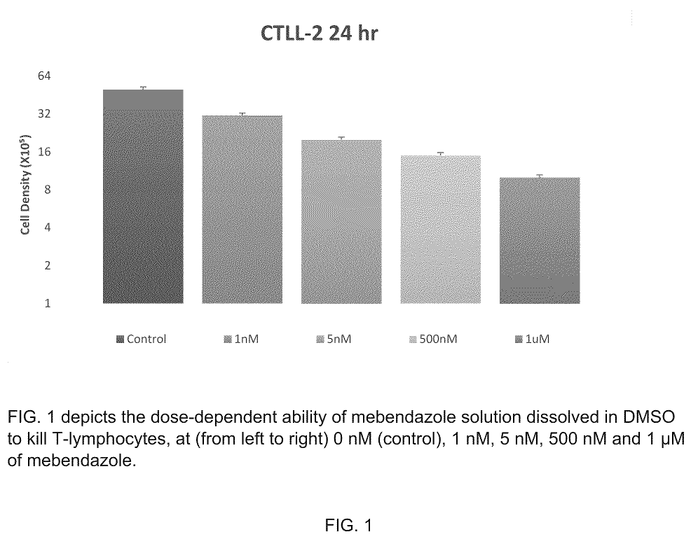

The mebendazole topical treatment compositions according to preferred embodiments of the invention are effective in the reduction of T-lymphocytes (see FIGS. 1-3), and can be used as effective immune modulators alone, as well as in combination with an additional therapeutic agent, such as oxymetazoline. Accordingly, disclosed herein are methods of treating or preventing a disease or disorder in a subject (preferably a human), comprising administering to the subject a therapeutically effective amount of a composition comprising one or more benzimidazoles in a vehicle or carrier for topical application. Most preferably, the benzimidazole comprises mebendazole, which is in a topical cream vehicle as described herein. Most preferably, the vehicle or carrier is a pharmaceutically acceptable carrier composition suitable to use on human and/or animal (non-human) skin. Preferably, the disease or disorder is an autoimmune disease or an inflammatory skin disorder. Most preferably, the disease or disorder is rosacea.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated.

As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a composition of the disclosure to an individual in need of such treatment. Within the meaning of the disclosure, “treatment” also includes relapse prophylaxis or phase prophylaxis, as well as the treatment of acute or chronic signs, symptoms and/or malfunctions. The treatment can be orientated symptomatically, for example, to suppress symptoms. It can be affected over a short period, be oriented over a medium term, or can be a long-term treatment, for example within the context of a maintenance therapy. As used herein, the terms “prevent,” “preventing,” and “prevention,” are art-recognized, and when used in relation to a condition, such as rosacea, is well understood in the art, and includes administration of a composition which reduces the frequency of, or delays the onset of, symptoms of a medical condition in a subject relative to a subject which does not receive the composition. Thus, prevention of rosacea includes, for example, reducing inflammation, restoring or reducing the levels of T-cell lymphocytes near hair follicles in a treated population versus an untreated control population, e.g., by a statistically and/or clinically significant amount.

In some cases, the compositions and methods disclosed herein comprise those for treating or preventing rosacea. In some cases, the compositions and methods disclosed herein comprise those for treating rosacea. In some cases, the compositions and methods disclosed herein comprise those for preventing rosacea.

Preferred MBZ treatment compositions comprise around 0.01-1.0% mebendazole by weight, more preferably around 0.05-0.5%, and most preferably around 0.1-0.15%. Depending on the other ingredients used in MBZ treatment compositions, particularly the solvents or penetrants used, the amount of mebendazole may be increased to up to around 20.0% by weight, more preferably up to around 10.0%, and most preferably up to around 5.0% to allow for sufficient solubility and skin penetration of the mebendazole. The balance of preferred MBZ treatment composition comprise pharmaceutically acceptable carrier or vehicle ingredients for topical administration and optionally one or more other pharmaceutically active ingredients. A pharmaceutically active vehicle or carrier may comprise a lotion, cream, solution, suspension, or ointment that is aqueous or non-aqueous, or a clear aqueous or anhydrous solution or gel. One preferred vehicle is a moisturizing cream or lotion comprising an emulsion or microemulsion for facial treatment or prevention of erythematotelangiectactic rosacea, papulopustular rosacea, and phymatous rosacea, and a sterile ophthalmic solution, suspension, emulsion, or ointment for treatment or prevention of ocular rosacea.

In some cases, the methods disclosed herein comprise administering to the subject one or more additional pharmaceutically active agents and/or such one or more additional pharmaceutically active agents may be included in the composition according to preferred embodiments. In some cases, the additional pharmaceutically active agent is an immunosuppressant, an anti-infective, calcineurin inhibitor, Janus kinase (JAK) inhibitor, retinoid or vasoconstricting agent. In some cases, the immunosuppressant is a corticosteroid. In some cases, the corticosteroid is hydrocortisone, triamcinolone, clobetasol, fluocinonide, etc. In some cases, the anti-infective agent is tetracycline, doxycycline, minocycline, erythromycin, metronidazole, ivermectin, etc. In some cases, the calcineurin inhibitor is cyclosporine, pimecrolimus or tacrolimus. In some cases, the JAK inhibitor is ruxolitinib, tofacitinib, baricitinib or oclacitinib. In some cases, the retinoid agent is azelaic acid, tretinoin, retinol. In some cases, the vasoconstricting agent is brimonidine, midodrine or oxymetazoline. In other preferred embodiments, any of these ingredients may also be excluded from the composition or excluded from use in the methods of the invention. Chronic use of corticosteroids are excluded from the composition according to some preferred embodiments as contraindicated.

Additionally, as the compositions and methods of preferred embodiment involve topical application to the skin or ocular membranes, other non-active excipients which may aid in or induce penetration of the active benzimidazole agent(s) into the facial skin or ocular tissue may be used. Substances termed “permeation enhancers,” are typically used in compositions designed to deliver drugs transdermally to increase the amount of the active that is delivered into the systemic circulation. Permeation enhancers constitute various classes of compounds including certain compounds such as dimethylsulfoxide, methylsulfonylmethane, pyrrolidones, ethanol, propylene glycol, dimethylacetamide, and others that are capable of disrupting the barrier function of the stratum corneum. Other substances have also been shown to increase the flux of certain active agents through the skin or mucous membranes. These include lipophilic compounds such as laurocapram (Azone); alpha-hydroxy and beta-hydroxy acids, fatty acids or alcohols such as oleic acid, oleyl alcohol, linoleic acid and the like; certain fatty acid esters such as isopropyl myristate, methyl nonanoate, methyl caprate and others. Mixtures of certain permeation enhancers with propylene glycol, butylene glycol, pentylene glycol, isopentyl glycol, hexylene glycol, diethylene glycol monoethyl ether; also known as ethoxydiglycol, acetamide MEA, and propylene glycol monolaurate are also known to improve the delivery of certain active ingredients. Certain surfactants may also increase penetration of active agents through solubilization and reduction in interfacial surface tension or encapsulation of the active agent and may include PEG-40 hydrogenated castor oil, Cremophor® or other surfactants combining glyceryl polyethylene glycol oxystearate, fatty acid glyceryl polyglyceryl esters, polyethylene glycol, and glyceryl ethoxylates. Encapsulation media for the benzimidazole drug substance or compositions according to preferred embodiments may also include soy or egg based lecithin or chemically modified lecithin, phosphatidylcholine or modified phosphatidylcholine, beta-cyclodextrins, PLGA [poly(lactic-co-glycolic acid], dextran, chitosan, d-alpha-tocopheryl polyethylene glycol succinate polymers and poloxamer (polyoxyethylene-polyoxypropylene) block polymers. Compositions according to preferred embodiments may comprise one or more of these ingredients or other permeation enhancers. In other preferred embodiments, any of these ingredients may also be excluded from the composition or excluded from use in the methods of the invention.

Although numerous ingredients may be used with the compositions according to certain preferred embodiments, according to other preferred embodiments they are excluded. Compositions according to certain preferred embodiments do not include NSAIDs, 2,6-di-tert-butyl-4-methylphenol, phenyl-pentanedione/or pyridine/or phenylbenzene/or benzoxazine compounds (such as 3-(4-methozyphenyl)-1,5-bis(2-methoxyphenyl)-1,5-pentanedione). Hyaluronic acid/salt may be included in certain preferred compositions and may be excluded in other preferred compositions according to the invention.

With respect to the amount of mebendazole incorporated in each topical formulation, the mebendazole content will typically be adjusted such that when the topical formulation is applied to a treatment area of a subject in need thereof, the amount of compound for reducing inflammation. (i.e., for treating rosacea) is present in an amount effective to achieve at least one of: (i) reduction of T-cell viability, (ii) reduction of T-cell proliferation, (iii) reduction of T-cell activation, (iv) reduction of T-cell activity.

Uses of the topical compositions disclosed herein in the preparation of a medicament for treating the diseases and disorders described herein are provided.

EXAMPLES. Described in the below Examples 1 and 2 are methods for evaluating mebendazole for efficacy in modulating T-lymphocytes in an in vitro platform translatable to human skin disease and its clinical application with a MBZ treatment composition according to a preferred embodiment of the invention. The results of these examples demonstrate that mebendazole is a promising immunomodulatory agent for treatment of rosacea and possibly other forms of inflammatory and neoplastic skin diseases driven by T-lymphocytes to ultimately improve the patient's quality of life. Described in the below Examples 3-7 are compositions and methods for making compositions comprising MBZ according to preferred embodiments of the invention, along with solubility analysis of MBZ in select solvents and Skin PAMPA testing of select compositions for evaluation of skin penetration. The following examples are provided for illustration and are not intended to limit the scope of the disclosure.

### Example 1—In Vitro Trial

An in vitro screening platform for immunomodulation was used to evaluate mebenzadole for efficacy. CTLL-2 (ATCC® TIB-214™) are cytotoxic T lymphocytes that were purchased and cultured according to ATCC protocol. Once confluent, cells were seeded in a 12-well plate at a concentration of 55,000 cells/mL and treated, in triplicates, with a solution of mebendazole dissolved in dimethyl sulfoxide (DMSO) at varying mebendazole concentrations of 1 nM, 5 nM, 500 nM and 1 μM for comparison to a control that had only DMSO applied (without any mebendazole). The maximum volume added to the 1 mL of each of the 12 wells was 10 μL for the highest test concentration of mebenzadole and the control. Although the concentrations of mebendazole are within preferred embodiments of treatment compositions according to the invention, the preferred treatment compositions for use do not include DMSO. MBZ has good solubility in DMSO (approximately 2% by weight), which is why it was selected as a solvent for this example; however, it is preferred to not use DMSO as a solvent in MBZ treatment compositions according to the invention due to safety considerations with skin applications. As indicated in FIG. 1, mebendazole is shown to dose dependently kill T-lymphocytes. The results indicate that mebenzadole significantly reduces the T-lymphocytes, in cell culture, starting at a test concentration of 1 nM (0.295 micrograms).

In vitro data disclosed in above shows significant reduction of inflammatory CD+8 T cells starting at concentrations of 1 nM or 0.295 μg per mL in DMSO. This extremely low concentration for reduction of T-cells becomes important in formulating mebendazole (or methyl 5-benzoylbenzimidazole-2-carbamate) in a topical cream, lotion, gel, suspension, emulsion, patch or any topical pharmaceutical vehicle for application to human or mammalian skin and most preferably to human facial skin afflicted with rosacea.

### Example 2—Application of Mebendazole for the Treatment of Rosacea

Without wishing to be bound by theory in the immune model of rosacea, the inflammatory process induced by activated T-cells leads to the following changes that give rise to all sub-types of rosacea previously noted: angiogenesis, formation of papules and pustules, and granulomatous inflammation that leads to phymatous changes of the skin. The recovery process is complex. Again, without wishing to be bound by theory, the activated T-cells must be deactivated (e.g., with mebendazole) and then the skin achieves homeostatic normalcy that resolves the cutaneous erythema, telangiectasias, papules, pustules and phymatous changes.

A clinical study was conducted to determine the effect of 10% mebendazole cream according to one preferred MBZ treatment composition applied daily according to one preferred treatment method on the remission of rosacea after 12 weeks. In order to collect the preliminary information on the remission of rosacea, a split-face study was performed where the Patient applied the MBZ treatment composition (comprising 10% mebendazole by weight) to the left side of her face and the vehicle cream (as a control, without any mebendazole) to the right side of her face nightly for 12 weeks.

The Patient is a 52-year-old female with clinically diagnosed papulopustular rosacea (PPR) of the face over decades. She received numerous treatments including topical ivermectin, topical metronidazole, topical steroids, topical antibiotics and systemic antibiotics. After discontinuation of the aforementioned treatments, her rosacea would flare with erythema, telangiectasias, papules and pustules. The Patient underwent a 4-week washout period without use of topical or systemic active medications prior to initiating the active (10% mebendazole) and control (vehicle) creams. The amount of mebendazole in the cream used in Example 2 is equivalent to a molar concentration of 0.339M and approximately 0.25 to 0.5 mL of the cream was applied to the left cheek of the Patient once per day.

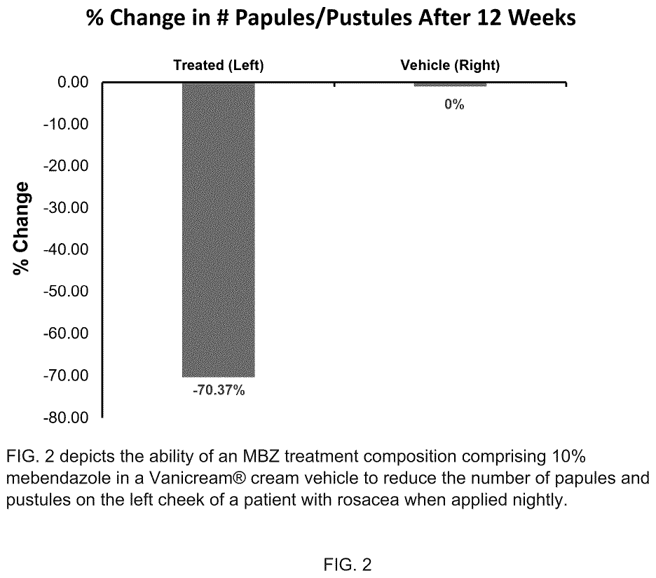

The results are summarized in FIG. 2. Reported categories are as follows: treated (10% mebendazole, left cheek) and control (vehicle, right cheek). In these results, active (10% mebendazole) showed a decrease in the total number of papules and pustules of the left face (70.37%) whereas the vehicle did not result in a decrease, indicating an improvement of the rosacea when treated according to a preferred embodiment of the invention.

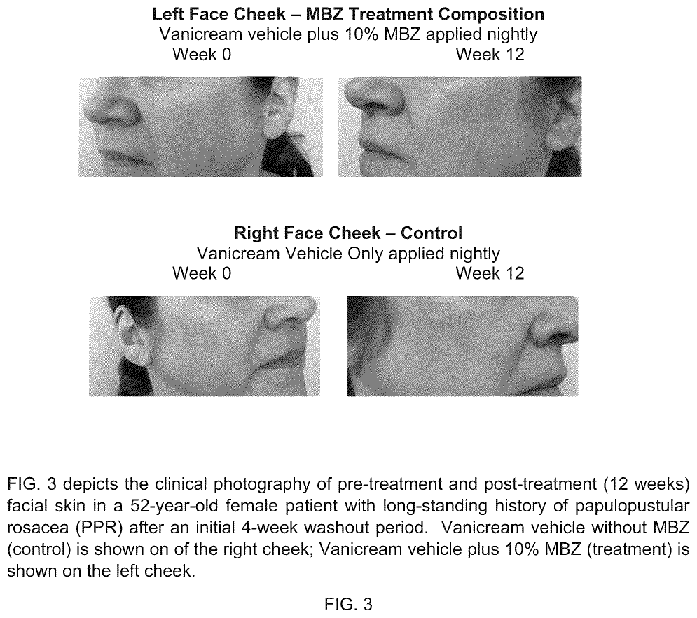

The results are also represented by photos in FIG. 3 depicting improvement of the rosacea of the left cheek with a 12-week nightly treatment with active (10% mebendazole) cream but no improvement of rosacea of the right cheek with 12-week nightly treatment with control (vehicle). Improvement is noted by reduction in papules and pustules as well as reduction in erythema and telangiectasias.

The MBZ treatment composition used in the treatment of the Patient was formulated by adding Mebenzadole Tablets, USP, 500 mg, crushed to a fine powder, mixed until uniform and in a sufficient amount to make a 10% by weight mebenzadole suspension in a commercially available Vanicream® Moisturizing Skin Cream vehicle. No attempt to increase the solubility of the MBZ in the commercial Vanicream® vehicle was made. The label ingredient listing (INCI; International Nomenclature Cosmetic Ingredient) for the Vanicream® Moisturizing Skin Cream, which is an oil-in-water emulsion cream base, is as follows: Purified water, petrolatum, sorbitol, cetearyl alcohol, propylene glycol, ceteareth-20 (a polyethylene glycol ether of cetearyl alcohol), simethicone, glyceryl stearate, PEG-30 Stearate (a polyethylene glycol ester of stearic acid), sorbic acid, BHT (butylated hydroxytoluene). MBZ is poorly soluble in water and it should be noted that the aqueous solubility of MBZ is approximately 0.035 mg/mL (Polymorph C at 25 degrees C.). In addition to negligible solubility in water, MBZ has negligible solubility in most solvents other than DMSO (approximately 2% by weight). MBZ is also freely soluble in formic acid. Neither DMSO nor formic acid is an appropriate pharmaceutical vehicle ingredient for a facial treatment product due to safety considerations. Thus, there is a need for a process to increase the solubility of MBZ in a pharmaceutical vehicle that would be appropriate for use on human and animal skin, particularly human facial skin.

### Example 3—Increased Penetration and Solubility Trials

Further laboratory experiments were conducted to increase the skin penetration of the MBZ by determining the maximum solubility of MBZ in certain topical pharmaceutical skin penetrants as shown in Table 1 below. The maximum solubility of MBZ was tested in the various preferred solvent ingredients and combinations of ingredients, specifically, (1) dimethyl isosorbide (solvent composition 1), (2) diethylene glycol monoethyl ether (Transcutol P®) (solvent composition 2), (3) propylene glycol monolaurate (Lauryl Glycol®) (solvent composition 3), and (4) combinations of Transcutol P and Lauryl Glycol in a weight ratio of around 60:40 (solvent composition 4) were evaluated by adding 1% by weight of MBZ to each of them in the formulas listed in Table 1. All formulas were mixed at 20-25° C. for 24 hours with magnetic stirring followed by filtering through a 0.45 micron (μ) membrane filter to remove undissolved solid particles of MBZ larger than 0.45 microns. Second batches of solvent compositions 1 (dimethyl isosorbide) and 2 (diethylene glycol monoethyl ether (Transcutol P®)) were also made and were further processed by exposure to a temperature of 100° C. for 15 minutes after filtering to form solvent compositions 5 and 6, respectively. All filtered formulas were crystal clear, except formulas 1 and 5 containing dimethyl isosorbide, which appeared milky white after filtration indicating solid MBZ with particle size less than 0.45μ. This visual appearance indicates that solvent compositions 2-4 and 6 had better solubility than solvent compositions 1 and 5 (with dimethyl isosorbide or DMI) because undissolved, but small, solid particles remained in solvent compositions 1 and 5; however, each of the solvent compositions were assayed by UV-VIS spectrophotometric (300-325 nm) or by HPLC (Mebendazole; USP-NF Monograph) methods to determine the amount of MBZ in the compositions after the filtration step.

As shown in Table 1, the results of the two assay methods are in general agreement except for the solvent compositions 5 and 6 made with exposure to 100° C. temperature for 15 minutes, which show lower assay results with the HPLC assay indicating possible MBZ degradation due to the additional heating step.

TABLE 1
Saturation Solubility of MBZ in Solvent-Penetrants

| | UV-VIS Assay | HPLC Assay; |
|---|---|---|
| Solvent Composition | MBZ content | MBZ content |
| (with MBZ) | (ug/mL) | (ug) |
| 1 (Dimethyl Isosorbide)* | 5048 | 4870 |
| 2 (Transcutol P) | 2386 | 2390 |
| 3 (Propylene Glycol | 379 | 460 |
| Monolaurate) | | |
| 4 (Transcutol P/Propylene | 833 | 930 |
| Glycol Monolaurate 60:40 | | |
| w/w) | | |
| 5 (Dimethyl Isosorbide; | 4968 | 2700 |
| heated to 100° C. for 15 | | |
| minutes)* | | |
| 6 (Transcutol P; heated | 4188 | 3560 |
| 100° C. for 15 minutes) | | |

*Milky after filtration

The solubility of MBZ was found to be highest in either solvent composition 1 (DMI) or 2 (Transcutol P). However, this solubility is reduced to 10-20% of the theoretical maximum amount when the solvent compositions 1 and 2 are diluted 50% with deionized water. Although concentrations of up to 0.5% by weight MBZ can be achieved in undiluted solvent compositions containing (1) only MBZ and Transcutol P or (2) only MBZ and DMI, the resulting liquid solvent composition has undesirable cosmetic or aesthetic qualities, including a lingering oiliness on skin application. These issues can be avoided by diluting such solvent compositions with water (preferably deionized or purified water); however, the diluted solvent composition can be runny, making it difficult to apply to skin, particularly facial skin. These issues can be avoided with appropriate viscosity modifiers or gelling agents to create an MBZ treatment composition comprising a topical gel or thickened liquid. Such viscosity modifiers or gelling agents may be part of a topical application vehicle to which MBZ (or an MBZ concentrate composition) is added or may be part of an MBZ gel composition further described herein.

### Example 4—Mixed Solvent Composition Trials

Further laboratory experiments were conducted to evaluate a combination of Trancutol P and DMI as solvents for MBZ, to maximize MBZ solubility and skin penetration. DMI and Transcutol P were used at levels shown to be safe for application to human skin and diluted with deionized water to a concentration of 40% by weight Transcutol P and a concentration of 15% DMI for these trials, with the addition of 0.05-0.2% by weight MBZ, and the balance deionized water. Deionized water was used as a diluent in these trials, to effectively substitute for a topical application vehicle, such as a preferred embodiment of an aqueous MBZ cream base composition further described herein.

Experimentation with the blend of 40% w/w Transcutol P, 15% DMI and 43-44% deionized water and concentrations of MBZ at 0.05-0.2% by weight required heating of the solvent, water, and MBZ mixture to effect more complete solubility of the MBZ. To keep temperatures as low as possible to avoid degradation of the MBZ, the solvent/water/MBZ mixtures were first stirred at 50° C. for 18 hours and then stirred at 60° C. for 18 hours. Test results showed some haziness of incomplete solubility and a color shift to yellow that was not observed with exposure of similar formulations heated to higher temperatures (90-100° C.) but for shorter times (5-15 minutes) such as compositions 5-6 in Example 3 (Table 1). Additionally, trials also showed that use of MBZ amounts higher than 0.15% by weight in the Transcutol P/DMI/deionized water (40/15/45% w/w) and MBZ solutions that were initially clear (indicating good solubility of the MBZ) could generate a more pronounced hazy precipitate after overnight storage. Based on these results, further formulation development testing on MBZ concentrations at 0.05-0.2% w/w and with heating at 60-90° C. for 5-15 minutes prior to adding the dilution water was conducted.

In these tests, it was found that the addition of heat to the MBZ/DMI/Transcutol P solvent mixture, prior to dilution with deionized water, solubility of MBZ increases in the final diluted composition or when mixed with an aqueous base vehicle, such as a preferred embodiment of an MBZ cream base composition herein. With heating between 60° C. and 90° C., more completely solubility of MBZ at 0.05-0.15% by weight can be achieved with mixing for 5-15 minutes. At 60 and 70° C., complete solubility of MBZ is achieved at a concentration of 0.05% by weight. As the concentration is increased above 0.05% by weight, 0.10% w/w MBZ requires 80° C. and 0.15% w/w MBZ requires 90° C.

Thus, according to one preferred embodiment, an MBZ concentrate composition is made by: (1) mixing an ethylene or propylene glycol based solvent (preferably diethylene glycol monoethyl ether (Transcutol P®)) with a sorbitol based solvent (preferably dimethyl isosorbide) and 0.05-0.2% w/w MBZ (more preferably 0.05-0.15% w/w MBZ); (2) heating to a temperature within a first temperature range; and (3) mixing or stirring during the heating step, such as by using a magnetic stirrer. Preferably, the first period of time is around 5 to 20 minutes, more preferably around 5 to 15 minutes. Most preferably, the heating temperature and first period of time will not result in degradation of the MBZ of more than 10%, more preferably not more than 8% and most preferably not more than 5%. The first temperature range is most preferably between at least 60° C. but less than a temperature at which MBZ will experience degradation of 5% or more. More preferably, the first temperature range is 60-90° C., and most preferably 70-90° C. Preferably, steps 1-3 are carried out before mixing the MBZ with any water or aqueous based application vehicle.

### Example 5—Trial Formulations

In order to potentially increase the vehicle solubility and skin penetration of MBZ, MBZ concentrate compositions according to preferred embodiments of the invention were prepared and added to a topical MBZ cream base (or vehicle) formulation according to a preferred embodiment of the invention for testing (as further described in Example 6). Preferred embodiments of MBZ concentrate compositions are shown in Table 2 and preferred embodiments of a topical MBZ cream base compositions are shown in Table 3. According to one preferred embodiment, an MBZ solvent/skin penetrant concentrate composition (formula 45-147) was prepared as follows: (1) MBZ was micronized to a an average particle size of around 3.5 to 4.0 microns; (2) the micronized MBZ powder is added to a liquid mixture of (a) a sorbitol based solvent (preferably dimethyl isosorbide), (b) an ethylene and/or propylene glycol based solvent (preferably diethylene glycol monoethyl ether (Transcutol P®), propylene glycol monolaurate (Lauryl Glycol®) 60:40), and (c) a solubilizer and/or emulsifying agent (preferably Cremophor RH 40/polyoxyl 40 hydrogenated castor oil); and (3) the MBZ powder is mixed with the liquid ingredients preferably at 25-50° C., more preferably at 35-50° C., and for 15-30 minutes or until a uniform white suspension of MBZ powder in the liquid is created.

TABLE 2
Mebendazole Solvent and Penetrant Concentrate

| | | | Most |
|---|---|---|---|
| MBZ CONCENTRATE COMPOSITION (45-147) | | Preferred | Preferred |
| | TRIAL Ex. 6 | Range | Range |
| | PERCENT | PERCENT | PERCENT |
| INGREDIENTS | (w/w)* | (w/w) | (w/w) |
| Sorbitol based Solvent (DIMETHYL ISOSORBIDE) | 38.30 | 32.00-45.00 | 36.00-40.00 |
| MEBENDAZOLE USP | 3.60 | 1.00-10.00 | 3.00-6.00 |
| Ethylene or propylene glycol based solvent | 19.85 | 15.00-25.00 | 18.00-21.00 |
| (DIETHYLENE GLYCOL MONOETHYL ETHER | | | |
| (TRANSCUTOL P)) | | | |
| Solubilizer and/or emulsifying agent (POLYOXYL | 38.25 | 32.00-45.00 | 36.00-40.00 |
| 40 HYDROGENATED CASTOR OIL (CREMOPHOR RH 40)) | | | |
| TOTAL | 100.00 | | |

*Amounts of a preferred embodiment used in the MBZ concentrate composition used to prepare MBZ treatment compositions for Example 6 testing.

A laboratory batch of 1 Kg of MBZ concentrate composition (formula 45-147) was made according to the Trial Example 6 percentages in Table 2 and divided into two parts substantially equal parts for use in preparing MBZ treatment compositions for use in Example 6. The first part of the concentrate batch is processed at 35-35° C. through a Microfluidics EH-110 microfluidizer at 14,000 psi for 3 discreet complete passes, while the second half of the concentrate batch is not processed further. These concentrate batches were then mixed with an MBZ cream base (formula 45-149, according to the Trial Example 6 percentages in Table 3) in proportions that would create final MBZ treatment compositions containing 0.05%, 0.1%, 0.25%, 0.5% and 1% MBZ in the cream base vehicle (with ingredient amounts as shown in Table 4).

The topical MBZ cream base composition (also referred to as formulation 45-149) is an oil-in-water (O/W) type cream emulsion base vehicle, preferred embodiments of which are shown in Table 3.

TABLE 3
Mebendazole Vehicle Cream Base

| | | | Most |
|---|---|---|---|
| MBZ CREAM BASE 45-149 | | Preferred | Preferred |
| | TRIAL Ex. 6 | Range | Range |
| | PERCENT | PERCENT | PERCENT |
| INGREDIENTS | (w/w)* | (w/w) | (w/w) |
| WATER | 86.45 | 80.00-95.00 | 83.00-85.00 |
| GLYCERIN, USP | 2.00 | 1.00-4.00 | 1.50-2.50 |
| CARBOMER | 0.50 | 0.1-1.0 | 0.2-0.6 |
| DISODIUM EDTA | 0.05 | 0.02-0.20 | 0.05-0.1 |
| CETYL ALCOHOL | 2.00 | 1.00-4.00 | 1.50-2.50 |
| STEARYL ALCOHOL | 1.50 | 1.00-3.00 | 1.25-1.75 |
| GLYCERYL STERATE | 4.50 | 2.00-6.00 | 4.00-5.00 |
| (and) PEG 100 STEARATE | | | |
| POLYSORBATE 80 | 1.75 | 1.00-2.50 | 1.50-2.00 |
| TRIETHANOLAMINE 99% | 0.50 | 0.25-1.5 | 0.50-1.00 |
| PHENOXYETHANOL | 0.75 | 0.25-1.00 | 0.50-0.800 |
| TOTAL | 100.00 | | |

*Amounts of a preferred embodiment used in the MBZ Base Cream composition used to prepare MBZ treatment compositions for Example 6 testing.

The MBZ cream base preferably comprises an aqueous phase composition and an oil phase composition. The aqueous phase composition is preferably made as follows: (1) mix the glycerin and phenoxyethanol together until a clear uniform solution is created; (2) add the deionized water to the solution with mixing; (3) heat the aqueous mixture to 60-70° C., while continuing to mix/stir; (4) add the disodium EDTA with mixing until dissolved in the water phase; (5) slowly add the Carbomer 940 to the heated water phase by sprinkling on the water surface and mix for 45 minutes (maintaining temperature at 60-70° C.) until a uniform hydrated dispersion of the carbomer is created. The oil phase composition is preferably separately made as follows: (1) mix the (a) cetyl alcohol, (b) stearyl alcohol, (c) glyceryl stearate (and) PEG 100 stearate (which is an emulsifier composition), and (d) polysorbate 80 together; (2) heat the mixture to 60-70° C. with mixing until a uniform liquid oil phase is created. The MBZ cream base is preferably made by (1) adding the aqueous phase composition and the oil phase composition together; (2) heat to 60-70° C. with homogenization (Silverson L4RT-A homogenizer) at 5000-7000 rpm for 15-30 minutes; (3) cool to around 35-50° C., more preferably around 35 OC, and add the triethanolamine while mixing until a smooth and uniform mixture is formed; and (4) further cool to around 25 OC (around room temperature). As further described below, it is most preferred that the MBZ concentrate composition be added and mixed with the MBZ cream base composition prior to cooling in step (4).

The final MBZ treatment compositions were made by mixing the required amount of MBZ concentrate composition, previously heated to 35-50° C., with the required amount of MBZ cream base composition, also previously heated to 35-50° C., in the desired proportions, followed by cooling to room temperature.

Table 4 shows the amounts of ingredients for the MBZ cream base as added to varying amounts of MBZ concentrate composition to create a range of MBZ topical treatment compositions A-F having 0.05% up to 1% by weight MBZ.

TABLE 4
Mebendazole Treatment Compositions

| | A | B | C | D | E |
|---|---|---|---|---|---|
| MBZ Treatment Compositions | | | | | |
| (Ex. 6 Test Formulations) | | | | | |
| MBZ Cream Base 45-149 Percent | 98.61 | 97.22 | 93.05 | 86.10 | 72.20 |
| MBZ Concentrate Phase 45-147 | 1.39 | 2.78 | 6.95 | 13.900 | 27.800 |
| Percent | | | | | |
| Individual Ingredient Percentages | | | | | |
| in MBZ Treatment Compositions | | | | | |
| WATER | 85.25 | 84.05 | 80.44 | 74.43 | 62.42 |
| GLYCERIN USP | 1.97 | 1.94 | 1.86 | 1.72 | 1.44 |
| CARBOMER | 0.49 | 0.49 | 0.47 | 0.43 | 0.36 |
| DISODIUM EDTA | 0.05 | 0.05 | 0.05 | 0.04 | 0.04 |
| CETYL ALCOHOL | 1.97 | 1.94 | 1.86 | 1.72 | 1.44 |
| STEARYL ALCOHOL | 1.48 | 1.46 | 1.40 | 1.29 | 1.08 |
| GLYCERYL STERATE (and) | 4.44 | 4.37 | 4.19 | 3.87 | 3.25 |
| PEG 100 STEARATE | | | | | |
| POLYSORBATE 80 | 1.73 | 1.70 | 1.63 | 1.51 | 1.26 |
| TRIETHANOLAMINE 99% | 0.49 | 0.49 | 0.47 | 0.43 | 0.36 |
| PHENOXYETHANOL | 0.74 | 0.73 | 0.70 | 0.65 | 0.54 |
| DIMETHYL ISOSORBIDE | 0.53 | 1.06 | 2.66 | 5.32 | 10.65 |
| MEBENDAZOLE, USP | 0.05 | 0.10 | 0.25 | 0.50 | 1.00 |
| DIETHYLENE GLYCOL MONOETHYL | 0.28 | 0.55 | 1.38 | 2.76 | 5.52 |
| ETHER (TRANSCUTOL P) | | | | | |
| POLYOXYL 40 HYDROGENATED | 0.53 | 1.06 | 2.66 | 5.32 | 10.63 |
| CASTOR OIL (CREMOPHOR RH 40) | | | | | |
| Total | 100.00 | 100.00 | 100.00 | 100.00 | 100.00 |

### Example 6—Skin PAMPA Testing for Permeation of MBZ

The MBZ treatment compositions A-E from Example 5 were created twice where one set of the treatment compositions used the first half of the lab batch of MBZ concentrate 45-147 that had been processed through the microfluidizer as previously described (shown in Table 5 as batches A1-E1) and a second set of the treatment compositions used the second half of the lab batch of MBZ concentrate 45-147 that had not been processed through the microfluidizer (shown in Table 5 as batches A2-E2). These sets of compositions were used in testing in this Example 6.

MBZ treatment compositions A1-E1 (with microfluidization of the MBZ concentrate) and A2-E2 (without microfluidization) were evaluated for skin penetration using the Skin PAMPA—Parallel Artificial Membrane Permeability Assay—in vitro artificial skin model (Pion, Inc.). Another treatment composition comprising Vanicream® plus 10% by weight MBZ as used in Example 2 was also tested as a control for comparison to the MBZ treatment compositions using MBZ concentrate compositions and MBZ cream base compositions according to preferred embodiments of the invention. The Vanicream® plus 10% by weight MBZ is also considered an MBZ treatment composition according to a preferred embodiment of the invention, but for purposes of these tests was treated as the control. Table 5 lists the compositions tested in the Skin PAMPA skin model, where 150 microliters of each composition was applied to the Skin PAMPA artificial skin membrane and a PAMPA receptor (or acceptor media) fluid of 20% aqueous Hydroxypropyl-beta-cyclodextrin solution. For all trials, measurements were taken at 0.25, 0.5, 1, 6, and 24 hour intervals and the temperature was 32° C. In addition to the listed compositions, negative controls of the MBZ cream base (without MBZ) and Vanicream (without MBZ added) were also evaluated in the SKIN PAMPA tests to measure any interference in the UV assay of MBZ in the PAMPA acceptor plate solution. The UV absorbance of the negative control vehicles (Vanicream or MBZ cream base) was subtracted from the UV assay of their corresponding test compositions with MBZ. Table 6 lists the average permeated mass of MBZ through the artificial skin membrane, as well as the standard deviation, for each of the MBZ treatment compositions tested.

TABLE 5
Skin PAMPA Testing Compositions

| MBZ Treatment | | % API* |
|---|---|---|
| Composition | Process | (MBZ) |
| MBZ I.a 0.05%- A1 | Microfluidized | 0.05% |
| MBZ I.a 0.1% - B1 | | 0.1% |
| MBZ I.a 0.25% - C1 | | 0.25% |
| MBZ I.a 0.5% - D1 | | 0.5% |
| MBZ I.a 1% - E1 | | 1% |
| MBZ II.a 0.05% - A2 | Without | 0.05% |
| MBZ II.a 0.1% - B2 | Microfluidization | 0.1% |
| MBZ II.a 0.25% - C2 | | 0.25% |
| MBZ II.a 0.5% - D2 | | 0.5% |
| MBZ II.a 1% - E2 | | 1% |
| Vanicream plus 10% MBZ | N/A | 10% |

*% API is the percentage of active pharmaceutical ingredient (w/w)

Skin PAMPA Method Reference: Pharmaceutics 2021 Oct. 21; 13(11):1758. doi: 10.3390/pharmaceutics13111758.

TABLE 6
Average Permeated Mass of Mebendazole at each time
point/SKIN PAMPA

| | Average Permeated Mass MBZ* | | | | | | | | | |
|---|---|---|---|---|---|---|---|---|---|---|
| MBZ Treatment | 0.25 hr | | 0.5 hr | | 1 hour | | 6 hours | | 24 hours | |
| Composition | Avg. | SD | Avg. | SD | Avg. | SD | Avg. | SD | Avg. | SD |
| MBZ I.a 0.05%-A1 | 0.04 | 0.06 | 0.30 | 0.24 | 0.28 | 0.19 | 1.94 | 1.14 | 4.13 | 2.09 |
| MBZ I.a 0.1%-B1 | 0.01 | 0.05 | 0.15 | 0.26 | 0.19 | 0.19 | 2.31 | 0.68 | 4.83 | 1.95 |
| MBZ I.a 0.25%-C1 | 0.00 | 0.04 | 0.37 | 0.49 | 0.22 | 0.10 | 3.60 | 1.02 | 9.12 | 1.27 |
| MBZ I.a 0.5%-D1 | 0.03 | 0.17 | 0.22 | 0.51 | 0.26 | 0.15 | 4.68 | 0.25 | 13.00 | 1.95 |
| MBZ I.a 1%-E1 | −0.01 | 0.03 | 0.22 | 0.45 | 0.24 | 0.10 | 4.83 | 1.27 | 14.35 | 2.76 |
| MBZ II.a 0.05%-A2 | −0.03 | 0.03 | 0.18 | 0.43 | 0.20 | 0.18 | 1.84 | 0.67 | 4.04 | 1.29 |
| MBZ II.a 0.1%-B2 | −0.01 | 0.06 | 0.08 | 0.27 | 0.17 | 0,14 | 2.19 | 0.96 | 2.62 | 1.71 |
| MBZ II.a 0.25%-C2 | 0.00 | 0.06 | 0.22 | 0.48 | 0.23 | 0.08 | 2.54 | 0.93 | 6.24 | 1.42 |
| MBZ II.a 0.5%-D2 | 0.02 | 0.04 | 0.21 | 0.32 | 0.26 | 0.15 | 4.61 | 1.29 | 15.52 | 3.23 |
| MBZ II.a 1%-E2 | 0.03 | 0.08 | −0.02 | 0.23 | 0.24 | 0.18 | 3.48 | 1.00 | 14.80 | 2.09 |
| Vanicream plus 10% | −0.03 | 0.05 | 0.23 | 0.56 | 0.44 | 0.18 | 5.02 | 1.75 | 17.14 | 4.74 |
| MBZ | | | | | | | | | | |

*Total μg of MBZ in acceptor solution based on application of 150 μL MBZ Treatment Composition to 0.3 cm² membrane.

The summary results of the Skin PAMPA testing of the MBZ formulations indicate that permeation of mebendazole across the Skin PAMPA membrane is slow, and no mebendazole was observed in the acceptor plate at the 0.25, 0.5, and 1 hour time points. After 6 hours, mebendazole can be measured in the acceptor plate showing that it has permeated across the Skin PAMPA membrane, and a concentration dependency observed, with greater concentrations of sample within the vehicles resulting in a greater transmembrane flux.

At low treatment composition loading (lower concentrations or lower % API of MBZ of 0.05-0.25% MBZ), a slight increase in flux is observed in the microfluidization compositions (A1-E1) compared with the compositions without microfluidization (A2-E2). This indicates that the microfluidization process is beneficial in increasing dermal penetration at low concentrations of MBZ. Low treatment composition loadings are also observed to undergo a significant reduction in the rate of MBZ flux after 6 hours which may be due to depletion of Mebendazole within the composition. Without being bound by theory, it is believed that this depletion is most likely due to a change in the flux dynamics—as the amount of MBZ saturates the membrane and enters the acceptor the concentration gradient of soluble and insoluble MBZ changes—rather than oxidation or other reaction causing a reduction in measurable MBZ. Similar trends are observed with a higher treatment composition loading (0.5-1%), but the changes in flux and permeated amount are less pronounced. Increasing the loading (increasing concentration or the % API of MBZ of 0.5-1% MBZ) in the treatment composition appears to negate these observed differences, with loadings greater than or equal to 0.5%, including the Vanicream®, showing similar permeation between vehicle types (MBZ cream base or Vanicream® and with or without microfluidization of the MBZ concentrate) and consistent flux at both short and long timescales.

Mebendazole flux and permeated amount is observed to increase with an increase of treatment composition loading, although permeated amount does not appear to increase proportionally with concentration of MBZ in the treatment compositions. Additionally, although the amounts using Vanicream® as the base vehicle for MBZ are slightly higher than when using the MBZ cream base as the base vehicle, no significant increase can be observed between the MBZ cream base vehicles containing 0.5% or 1% MBZ and the Vanicream plus 10% MBZ control. Given that the Vanicream plus 10% MBZ control uses 10-20× the amount of MBZ compared to the 1% and 0.5% amounts in the treatment compositions using the MBZ concentrate (with its solvents designed to increase the solubility of the mebendazole and its penetration) in the MBZ cream base as the vehicle, there is a substantial improvement achieved through the use of the MBZ cream base according to preferred embodiments herein as they require substantially less active MBZ to achieve similar dermal penetrations.

Since topical skin treatment of rosacea will most likely be chronic daily use with a topical MBZ treatment composition according to preferred embodiments, the 24-hour cumulative penetration data for the Skin PAMPA test samples would seem to be most relevant. The 24-hour cumulative penetration of Mebendazole for the Vanicream plus 10% MBZ control were not statistically different than the 0.5% and 1% MBZ treatment compositions D1-E1, D2-E2 with a range of approximately 13-17 μg of MBZ, indicating that the skin penetrants and MBZ solvents used in the MBZ concentrate composition 45-147 most likely were responsible for increasing the MBZ penetration relative to the MBZ content of the D2-E2 and D1-E1 treatment compositions compared to the Vanicream plus 10% MBZ control. The microfluidization processing used for the MBZ concentrate 45-147 used in the D1-E1 treatment compositions appears to have had no significant effect on MBZ penetration over the longer term 24 hour test.

### Example 7—MBZ Concentrate Gel Compositions

For topical application to skin, it is advantageous to have a thickened or gel composition comprising MBZ. An MBZ gel composition is preferably one that can be directly applied to the skin with or without being pre-mixed with a separate vehicle or other vehicle ingredients, such as MBZ cream base compositions or Vanicream®. The use of the term “gel” is not intended to be limiting and includes thickened compositions, such as creams, ointments, and emulsions, preferably having a viscosity of around 10,000 to 400,000 centipoise, more preferably of around 50,000 to 200,000 centipoise, that are suitable for application to human and/or animal skin.

MBZ gel compositions according to preferred embodiments include MBZ in combination with one or more of the other ingredients as shown in Table 8.

TABLE 8
Mebendazole Gel Compositions

| | Preferred | Most Preferred |
|---|---|---|
| | Range | Range |
| | PERCENT | PERCENT |
| MBZ Gel Composition Ingredients | (w/w) | (w/w) |
| MEBENDAZOLE USP | 0.01-0.5 | 0.05-0.15 |
| Sorbitol based Solvent (preferably DIMETHYL ISOSORBIDE) | 5-25 | 10-15 |
| Ethylene or propylene glycol based solvent (preferably | 15-50 | 25-45 |
| DIETHYLENE GLYCOL MONOETHYL ETHER | | |
| (TRANSCUTOL P)) | | |
| Solubilizer and/or emulsifying agent (POLYOXYL 40 | 0-10 | 0-5 |
| HYDROGENATED CASTOR OIL (CREMOPHOR RH 40)) | | |
| Cyclodextrin compound, preferably hydroxypropyl beta- | 0-15 | 5-9 |
| cyclodextrin (Cavasol ® W7 HP) | | |
| Viscosity modifying agent (preferably Hydroxyethylcellulose | 0.5-3 | 1-2 |
| (Natrosol ® 250HR)) | | |
| Water (preferably deionized) | 20-70 | 35-50 |

Table 9 below lists preferred embodiments of MBZ gel compositions 45-173A through 45-173F. According to one preferred embodiment, these compositions were prepared and the dissolution of the MBZ in these compositions was achieved by: (1) moderate magnetic stirrer mixing of the Transcutol P and DMI and MBZ; (2) heating at the stated temperature (60, 70, 80, or 90° C.) for a first period of time of 5-15 minutes; (3) continuing to mix/stir during the heating step, such as by using the magnetic stirrer; and followed by (4) slow addition of water (preferably deionized), and any other ingredients (such as Natrosol gellant) at 35-50° C., while continuing to mix/stir to force cooling to room temperature within 15 minutes. For best solubility of the MBZ, it is important that steps (1)-(3) are carried out prior to the addition of water in step (4). According to other preferred embodiments, one or more other ingredients, such as the viscosity modifying agents or gelling agents may be pre-mixed or pre-dissolved in the water prior to step (4).

TABLE 9
Mebendazole Gel Formulations/Heat Processing

| | 45-173-A | 45-173-B | 45-173-C | 45-173-D | 45-173-E | 45-173-F |
|---|---|---|---|---|---|---|
| Ingredient | (60° C.) | (70° C.) | (80° C.) | (90° C.) | (90° C.) | (90° C.) |
| Mebendazole, USP | 0.1 | 0.1 | 0.1 | 0.1 | 0.15 | 0.20 |
| Dimethyl Isosorbide | 15 | 15 | 15 | 15 | 15 | 15 |
| DIETHYLENE GLYCOL | 40 | 40 | 40 | 40 | 40 | 40 |
| MONOETHYL ETHER | | | | | | |
| (Transcutol P) | | | | | | |
| Hydroxyethylcellulose | 1 | 1 | 1 | 1 | 1 | 1 |
| (Natrosol ® 250HR) | | | | | | |
| Deionized Water | 43.9 | 43.9 | 43.9 | 43.9 | 43.85 | 43.8 |
| Total (% w/w) | 100 | 100 | 100 | 100 | 100 | 100 |

To prevent precipitation of MBZ from the compositions in Tables 2, 8 or 9, the addition of 2-10% of a cyclodextrin compound (preferably hydroxypropyl beta-cyclodextrin (Cavasol® W7 HP)) may optionally, but preferably, be added to the compositions. If the cyclodextrin compound is used, it is preferably first dissolved in the deionized water prior to the addition of the viscosity modifying agent (preferably hydroxyethylcellulose (Natrosol)). According to other preferred embodiments, other viscosity modifying agents, such as carboxymethylcellulose, hydroxypropylcellulose, magnesium aluminum silicate, Carbomer, or other pharmaceutically acceptable viscosity modifying agents may be used in place of or in addition to hydroxyethylcellulose (Natrosol). Other ingredients that may improve or maintain stability of the compositions according to the invention, such as antioxidants, including ascorbic acid or ascorbate salts or ascorbyl esters, ferulic acid, ubidecarenone, dl-alpha tocopherol, BHT, BHA, or other pharmaceutically acceptable antioxidants may be added to the compositions according to preferred embodiments herein, as will be understood by those of ordinary skill in the art. Such antioxidant ingredients may be beneficial in improving or maintaining MBZ product stability.

Various other approaches have been made and published to increase the water solubility and the GI absorption of mebenzadole's use as an anthelminthic. Any known methods or compositions for increasing water solubility and/or dermal absorption of mebendazole may also be used with compositions and methods according to preferred embodiments of the invention.

According to still other preferred embodiments, topical compositions, MBZ concentrate compositions, and MBZ treatment compositions comprise ingredients and amounts according to one or more of the following paragraphs:

(A) A topical composition for treating or preventing an inflammatory or autoimmune disease or condition of human skin comprising 0.25 to 20% of one or more benzimidazole compounds by weight of the topical composition.

(B) The topical composition of paragraph (A) further comprising a carrier suitable for application to human skin and/or wherein the topical composition has a viscosity of around 10,000 to 400,000 centipoise and/or wherein the benzimidazole has a particle size of around 3.5 to 4.0 microns as an ingredient prior to any dissolution in the topical composition.

(C) The topical composition of paragraph (B) wherein the carrier comprises a cream, gel, lotion, liquid, emulsion, microemulsion, aerosol spray, non-aerosol spray, serum, solution, suspension, or ointment.

(D) The topical composition of paragraphs (B) or (C) wherein the carrier is non-aqueous.

(E) The topical composition of paragraphs (B) or (C) wherein the carrier is aqueous.

(F) The topical composition of any one of paragraphs (A)-(E) wherein the one or more benzimidazole compounds comprises mebendazole.

(G) The topical composition of any one of paragraphs (A)-(F) wherein the disease or condition comprises any form of rosacea.

(H) The topical composition of any one of paragraphs (A)-(G) wherein the one or more benzimidazole compounds comprises one or more of mebenzadole, fenbenzadole, albenzadole, and thiabenzadole.

(I) The topical composition of any one of paragraphs (A)-(H) wherein the topical composition comprises around 1-30% of a mebendazole concentrate composition and/or around 70-99% of vehicle or carrier composition suitable for application to human skin, the percentages by weight of the topical composition.

(J) The topical composition of paragraph (I) wherein the mebendazole concentrate composition comprises: (1) around 1-10% mebendazole; (2) around 32-45% of a sorbitol based solvent; (3) around 15-25% of a glycol based solvent comprising an ethylene glycol based solvent, or a propylene glycol based solvent, or both; and/or (4) around 32-45% total of a solubilizer or emulsifying agent or both; and wherein the percentages are by weight of the mebendazole concentrate.

(K) The topical composition of paragraph (J) wherein: the sorbitol based solvent comprises dimethyl isosorbide; the ethylene glycol based solvent comprises diethylene glycol monoethyl ether and/or the propylene glycol based solvent comprises propylene glycol monolaurate; and/or the solubilizer comprises polyoxyl 40 hydrogenated castor oil.

(L) The topical composition of any one of paragraphs (A)-(K) wherein the topical composition comprises around 0.01 to 1% mebendazole by weight of the topical composition.

(M) An aqueous mebendazole treatment composition for topically treating or preventing an inflammatory or autoimmune disease or condition of human skin, the treatment composition comprising: around 0.01-1% of one or more benzimidazole compounds; around 5-25% of a sorbitol based solvent; and/or around 15-50% of a glycol based solvent comprising an ethylene glycol based solvent, or a propylene glycol based solvent, or both; and wherein the percentages are by weight of the mebendazole treatment composition.

(N) The aqueous mebendazole treatment composition of paragraph (M) wherein: the one or more benzimidazole compounds comprises mebendazole; and/or the sorbitol based solvent comprises dimethyl isosorbide; and/or the ethylene glycol based solvent comprises diethylene glycol monoethyl ether; and/or the propylene glycol based solvent comprises propylene glycol monolaurate; and/or wherein the aqueous mebendazole composition has a viscosity of around 10,000 to 400,000 centipoise and/or wherein the mebendazole has a particle size of around 3.5 to 4.0 microns as an ingredient prior to any dissolution in the aqueous mebendazole composition.

(O) The aqueous mebendazole treatment composition of any one of paragraphs (M)-(N) further comprising: around 1-10% total of a solubilizer or emulsifying agent or both; around 1-15% of a cyclodextrin compound; and/or around 0.5-3% of a viscosity modifying agent.

(P) The aqueous mebendazole treatment composition of paragraph (O) wherein: the solubilizer comprises polyoxyl 40 hydrogenated castor oil; the cyclodextrin compound comprises hydroxypropyl beta-cyclodextrin; and/or the viscosity modifying agent comprises hydroxyethylcellulose.

(Q) The aqueous mebendazole treatment composition of any one of paragraphs (M)-(P) wherein the composition is a gel.

(R) The aqueous mebendazole treatment composition of any one of paragraphs (M)-® wherein the glycol based solvent is both the diethylene glycol monoethyl ether and the propylene glycol monolaurate in a weight ratio of around 55:45 to 65:35.

According to still other preferred embodiments, a method of treating or preventing an inflammatory or autoimmune disease or condition of human or animal skin comprises steps, ingredients, and amounts according to one or more of the following paragraphs:

(S) A method for treating or preventing an inflammatory or autoimmune disease or condition of human skin, the method comprising applying (1) a topical composition comprising 0.25 to 20% by weight of one or more benzimidazole compounds and a carrier or (2) a topical composition according to any one of paragraphs (A)-(L) or (3) an aqueous mebendazole treatment composition according to any one of paragraphs (M)-(R) to an area of a subject's skin affected by the disease or condition.

(T) The method of paragraph (S) wherein the carrier is aqueous.

(U) The method of paragraph (S) wherein the carrier is non-aqueous.

(V) The method of any one of paragraphs (S)-(U) wherein the carrier comprises a cream, gel, lotion, liquid, emulsion, microemulsion, aerosol spray, non-aerosol spray, serum, solution, suspension, or ointment for topical application to skin.

(W) The method of any one of paragraphs (S)-(V) wherein the one or more benzimidazole compounds comprises mebendazole and wherein the disease or condition comprises any form of rosacea.

(X) The method of any one of paragraphs (S)-(W) wherein the applying step is repeated at least once per day for a treatment period comprising at least two weeks.

(Y) The method of any one of paragraphs (S)-(X) wherein the applying step comprises applying around 0.025 to 0.5 g to each side of the facial skin affected by rosacea of the topical composition.

(Z) The method of any one of paragraphs (S)-(Y) wherein the method achieves a reduction in the number of cutaneous cytotoxic CD+8 T-cells of 50% or greater in the area of skin at the end of the treatment period compared to a number of cutaneous cytotoxic CD+8 T-cells in the area prior to the treatment period.

(AA) The method of any one of paragraphs (S)-(Z) wherein the method achieves a reduction of a number of papules or pustules or diffuse redness intensity of 25% or greater on the area of skin at the end of the treatment period compared a number of papules or pustules on the area prior to the treatment period.

(BB) The method of any one of paragraphs (S)-(AA) further comprising repeating the applying step at least once per day for a treatment period comprising at least twelve weeks; wherein the one or more benzimidazole compounds comprises mebendazole; wherein the carrier comprises a cream, gel, lotion, liquid, emulsion, microemulsion, aerosol spray, non-aerosol spray, serum, solution, suspension, or ointment for topical application to skin; wherein the disease or condition comprises any form of rosacea; and/or wherein the method achieves a reduction in the number of cutaneous cytotoxic CD+8 T-cells of 50% or greater in the area of skin at the end of the treatment period compared to a number of cutaneous cytotoxic CD+8 T-cells in the area prior to the treatment period.

According to still other preferred embodiments, a method of making a mebendazole treatment composition for topically treating or preventing an inflammatory or autoimmune disease or condition of human skin comprises steps, ingredients, and amounts according to one or more of the following paragraphs:

(CC) A method of making a mebendazole treatment composition for topically treating or preventing an inflammatory or autoimmune disease or condition of human skin, the method comprising: (1) adding an amount of one or more solvents and an amount mebendazole to form a first mixture; and (2) heating the first mixture to a first temperature within a first temperature range for a first period of time while mixing or stirring to form a heated mixture; and wherein the amount of mebendazole is around 0.05-0.2% by weight of the mebendazole treatment composition.

(DD) The method of paragraph (CC) wherein the first period of time is around 5 to 20 minutes; and/or wherein the first temperature range is at least 60° C. but less than a temperature at which mebendazole will experience degradation of 5% or more.

(EE) The method of any one of paragraphs (CC)-(DD) wherein the amount of mebendazole is 0.05-0.15%.

(FF) The method of any one of paragraphs (CC)-(EE) wherein the first temperature range is 60-90° C.

(GG) The method of any one of paragraphs (CC)-(EE) wherein the first temperature range is 70-90° C.

(HH) The method of any one of paragraphs (CC)-(GG) wherein the first period of time is 5 to 15 minutes.

(II) The method of any one of paragraphs (CC)-(HH) wherein the one or more solvents comprise a sorbitol based solvent, or a glycol based solvent or both.

(JJ) The method of paragraph (II) wherein: (1) the sorbitol based solvent comprises dimethyl isosorbide; and/or (2) the glycol based solvent comprises diethylene glycol monoethyl ether, propylene glycol monolaurate, or both.

(KK) The method of any one of paragraphs (CC)-(JJ) further comprising: cooling the heated composition to a second temperature within a second range of temperatures by adding water and mixing or stirring; and wherein the second range of temperatures is around 35-50° C.

(LL) The method of paragraph (KK) wherein the cooling step is completed in 15 minutes or less.

(MM) The method of any one of paragraphs (KK)-(LL) further comprising dissolving an amount of a viscosity modifying agent or an amount of an anti-precipitation agent or both in the water prior to the cooling step.

(NN) The method of paragraph (MM) wherein the viscosity modifying agent comprises hydroxyethylcellulose.

(OO) The method of any one of paragraphs (MM)-(NN) wherein the anti-precipitation agent comprises a cyclodextrin compound and the amount of the anti-precipitation agent is around 2-10% by weight of the mebendazole treatment composition.

(PP) The method of paragraph (OO) wherein cyclodextrin compound comprises hydroxypropyl beta-cyclodextrin.

(QQ) The method of any one of paragraphs (CC)-(PP) wherein the heated composition is microfluidized in a microfluidizer at 14,000 psi for at least one pass.

(RR) The method of paragraph (QQ) wherein the heated composition is microfluidized in a microfluidizer at 14,000 psi for at least three passes.

(SS) The method of any one of paragraphs (CC)-(PP) wherein the method does not comprise a microfluidization step.

(TT) The method of any one of paragraphs (CC)-(SS) wherein the mebendazole treatment composition has a viscosity of around 10,000 to 400,000 centipoise.

(UU) The method of any one of paragraphs (CC)-(TT) further comprising micronizing the amount of the mebendazole to a particle size of around 3.5 to 4.0 microns prior to the adding step.

(V V) The method of any one of paragraphs (CC), (EE), or (II)-(SS) wherein the first temperature range is 25-50° C. and the first time period is 15-30 minutes.

Preferred embodiments of compositions and methods according to the invention are capable of achieving one or more of the following benefits: (1) reducing T-lymphocyte cell density by at least around 50%, more preferably by at least around 90% compared to before treatment with a mebendazole composition; (2) reducing the number of papules and/or pustules or diffuse redness on the skin of a rosacea patient by at least around 25%, more preferably by at least around 75% compared to before treatment with a mebendazole composition; (3) reducing the appearance of swelling on the skin compared to before treatment with a mebendazole composition; (4) reducing a level of itchiness on the skin compared to before treatment with a mebendazole composition; and/or (5) for the treatment of erythematotelangiectactic rosacea, a significant reduction in the redness “a” or “a*” value of the facial skin is demonstrated with the topical mebenzadole treatment. The “a” or “a*” value is an objective measurement of redness as measured by the Hunter L,a,b (Hunter Labs) and CIE L*a*b* (CIELAB) colorimeters, the Minolta CR type colorimeters or other suitable colorimeter or chromameter. Visible changes in redness can be detected in colorimeter a* units as low as 0.2 units and a decrease in one (1.0) or more a or a* colorimeter units after topical treatment of erythematotelangiectactic rosacea is generally considered to show clinical efficacy.

Preferred embodiments of MBZ treatment compositions and methods according to the invention are also capable of achieving cumulative skin penetrations after 24 hours of at least 5 μg of MBZ, more preferably at least 10 μg, most preferably at least 15 μg. These amounts are as measured in acceptor solution using the Skin PAMPA tests described herein and based on application of 150 μL MBZ Treatment Composition to 0.3 cm² artificial skin membrane.

All numerical values, ratios, or percentages indicated herein as a range include each individual amount, numerical value, or ratio within those ranges and any and all subset combinations within ranges, including subsets that overlap from one preferred range to a more preferred range. Any ingredient other than the benzimidazole described as included in an embodiment herein may also be excluded from such embodiment. Unless specifically excluded, any preferred features and optional ingredients any composition embodiment and/or method steps described herein may be used with any other embodiment, even if not specifically described herein with that particular embodiment. References herein to water (without any modifier) include potable water, distilled water, deionized water, or other forms of purified, filtered, or cleaned water suitable for use in topical skin treatment compositions. These forms of water may be substituted for references herein to deionized water, other than in the claims. Those of ordinary skill in the art will also appreciate upon reading this specification, including the examples contained herein, that modifications and alterations to the preferred embodiments of a composition and its method of use may be made within the scope of the invention and it is intended that the scope of the invention disclosed herein be limited only by the broadest interpretation of the appended claims to which the inventor is legally entitled.

1. A topical composition for treating or preventing a disease or a condition of human skin comprising:
0.01 to 0.10% of one or more benzimidazole compounds;
wherein there are no active agents in the topical composition other than the one or more benzimidazole compounds;
wherein percentages are by weight of the topical composition; and
wherein the topical composition does not include any dimethyl sulfoxide and does not include any dimethylacetamide.

2. The topical composition of claim 1 further comprising:
a carrier suitable for application to human skin;
an ethylene glycol based solvent comprising diethylene glycol monoethyl ether; and
wherein the one or more benzimidazole compounds comprises mebendazole;
wherein the topical composition is suitable for application to an area of the human skin having the disease or the condition and wherein the disease or the condition is inflammatory or autoimmune in nature.

3. The topical composition of claim 2 wherein the carrier comprises a cream, gel, lotion, liquid, emulsion, microemulsion, aerosol spray, non-aerosol spray, serum, solution, suspension, or ointment; and
wherein there are no active agents in the topical composition other than the mebendazole.

4. The topical composition of claim 3 wherein the carrier is non-aqueous.

5. The topical composition of claim 3 wherein the carrier is aqueous;
wherein the mebendazole and the ethylene glycol based solvent are heated to 60° C. to 90° C. for 5 to 20 minutes to form a mebendazole solution;
wherein the topical composition comprises a mixture of the mebendazole solution and the carrier.

6. The topical composition of claim 1 wherein the one or more benzimidazole compounds and a solvent are part of a benzimidazole solution that is heated to a temperature in a temperature range of 60° C. to 90° C. for period of time of 5 to 20 minutes;
wherein the topical composition comprises the benzimidazole solution and a carrier;
wherein the solvent comprises diethylene glycol monoethyl ether; and
wherein the one or more benzimidazole compounds comprises mebendazole and wherein the disease or the condition comprises any form of rosacea.

7. The topical composition of claim 1 further comprising an ethylene glycol based solvent comprising diethylene glycol monoethyl ether; and
wherein the one or more benzimidazole compounds comprises mebenzadole.

8. The topical composition of claim 1 wherein the topical composition comprises around 1-30% of a mebendazole concentrate composition and around 70-99% of vehicle composition suitable for application to human skin, the percentages by weight of the topical composition.

9. The topical composition of claim 2 wherein the topical composition comprises:
around 0.15-5.5% of the diethylene glycol monoethyl ether; and
wherein the disease or the condition comprises any form of rosacea.

10. The topical composition of claim 9 wherein:
around 32-45% of a sorbitol based solvent comprising dimethyl isosorbide;
around 0.15-25% of a propylene glycol based solvent comprising propylene glycol monolaurate; and
around 32-45% of a solubilizer comprising polyoxyl 40 hydrogenated castor oil.

11. (canceled)

12. A method for treating or preventing a disease or a condition comprising applying a topical composition to an area of a subject's skin affected by the disease or the condition;
wherein the topical composition comprises a carrier and 0.01 to 0.10% of one or more benzimidazole compounds, wherein percentages are by weight of the topical composition; and
wherein the disease or the condition is inflammatory or autoimmune.

13. The method of claim 12 wherein the carrier is aqueous.

14. The method of claim 13 wherein the carrier comprises a cream, gel, lotion, liquid, emulsion, microemulsion, aerosol spray, non-aerosol spray, serum, solution, suspension, or ointment for topical application to human skin.

15. The method of claim 12 wherein the carrier is non-aqueous.

16. The method of claim 12 wherein the one or more benzimidazole compounds comprises mebendazole and wherein the disease or the condition comprises any form of rosacea.

17. The method of claim 16 wherein the applying step is repeated at least once per day for a treatment period comprising at least two weeks.

18. The method of claim 17 wherein the applying step comprises applying around 0.025 to 0.5 g of the topical composition to the area of the subject's skin affected by the rosacea.

19. The method of claim 18 wherein the area of the subject's skin has a number of cutaneous cytotoxic CD+8 T-cells and wherein a reduction of 50% or greater in the number of the cutaneous cytotoxic CD+8 T-cells in the area of the subject's skin to which topical composition is applied at an end of the treatment period is achieved compared to the number of cutaneous cytotoxic CD+8 T-cells in the area of the subject's skin prior to the treatment period.

20. The method of claim 18 wherein a reduction of 25% or greater in a number of papules or pustules or diffuse redness intensity on the area of the subjects skin at an end of the treatment period is achieved compared the number of the papules or the pustules or the diffuse redness intensity on the area of subject's skin prior to the treatment period.

21. The method of claim 18 further comprising repeating the applying step at least once per day for a treatment period comprising at least twelve weeks;
wherein the one or more benzimidazole compounds comprises mebendazole;
wherein the carrier comprises a cream, gel, lotion, liquid, emulsion, microemulsion, aerosol spray, non-aerosol spray, serum, solution, suspension, or ointment for topical application to skin;
wherein the disease or the condition comprises any form of rosacea; and
wherein a reduction of 50% or greater in a number of cutaneous cytotoxic CD+8 T-cells in the area of the subject's skin at an end of the treatment period is achieved compared to the number of cutaneous cytotoxic CD+8 T-cells in the area of the subject's skin prior to the treatment period.

22. A topical aqueous mebendazole treatment composition comprising:
an active ingredient suitable for treating or preventing a disease or a condition of human skin that is inflammatory or autoimmune, wherein the active ingredient corn rises around 0.01-0.10% of mebendazole;
an ethylene glycol based solvent comprising diethylene glycol monoethyl ether;
56% or more water; and
wherein percentages are by weight of the topical aqueous mebendazole treatment composition.

23. The topical aqueous mebendazole treatment composition of claim 22 further comprising:
dimethyl isosorbide; and
propylene glycol monolaurate.

24. The topical aqueous mebendazole treatment composition of claim 23 further comprising:
around 1-10% total of a solubilizer or a emulsifying agent or both;
around 1-15% of a cyclodextrin compound; and
around 0.5-3% of a viscosity modifying agent.

25. The topical aqueous mebendazole treatment composition of claim 24 wherein:
the solubilizer or the emulsifying agent comprises polyoxyl 40 hydrogenated castor oil;
the cyclodextrin compound comprises hydroxypropyl beta-cyclodextrin; and
the viscosity modifying agent comprises hydroxyethylcellulose.

26. The topical aqueous mebendazole treatment composition of claim 24 in a gel form.

27. The topical aqueous mebendazole treatment composition of claim 24 further comprising propylene glycol monolaurate and wherein the diethylene glycol monoethyl ether and the propylene glycol monolaurate are in a weight ratio of around 55:45 to 65:35.

28. A method of making a mebendazole treatment composition for topically treating or preventing a disease or a condition of human skin that is inflammatory or autoimmune, the method comprising:
(1) adding an amount of one or more solvents and an amount of mebendazole to form a first mixture; and
(2) heating the first mixture to a first temperature within a first temperature range for a first period of time while mixing or stirring to form a heated mixture; and
wherein the amount of mebendazole is around 0.05-0.10% by weight of the mebendazole treatment composition.

29. The method of claim 28 wherein the first period of time is around 5 to 20 minutes; and
wherein the first temperature range is at least 60° C. but less than a temperature at which mebendazole will experience degradation of 5% or more.

30. The method of claim 28 wherein the amount of mebendazole is 0.05-0.08%.

31. The method of claim 28 wherein the first temperature range is 60-90° C.

32. The method of claim 28 wherein the first temperature range is 70-90° C.

33. The method of claim 28 wherein the first period of time is 5 to 15 minutes.

34. The method of claim 28 wherein the one or more solvents comprise a sorbitol based solvent, or a glycol based solvent or both.

35. The method of claim 34 wherein:
the sorbitol based solvent comprises dimethyl isosorbide;
the glycol based solvent comprises diethylene glycol monoethyl ether, or propylene glycol monolaurate, or both.

36. The method of claim 35 further comprising:
cooling the heated mixture to a second temperature within a second range of temperatures by adding water and mixing or stirring; and
wherein the second range of temperatures is around 35-50° C.

37. The method of claim 36 wherein the cooling step is completed in 15 minutes or less.

38. The method of claim 36 further comprising dissolving an amount of a viscosity modifying agent or an amount of an anti-precipitation agent or both in the water prior to the cooling step.

39. The method of claim 38 wherein the viscosity modifying agent comprises hydroxyethylcellulose.

40. The method of claim 38 wherein the anti-precipitation agent comprises a cyclodextrin compound and the amount of the anti-precipitation agent is around 2-10% by weight of the mebendazole treatment composition.

41. The method of claim 40 wherein cyclodextrin compound comprises hydroxypropyl beta-cyclodextrin.

42. The method of claim 28 wherein the heated mixture is microfluidized in a microfluidizer at 14,000 psi for at least one pass.

43. The method of claim 42 wherein the heated mixture is microfluidized in a microfluidizer at 14,000 psi for at least three passes.

44. The method of claim 28 wherein the method does not comprise a microfluidization step.

45. The method of claim 28 wherein the mebendazole treatment composition has a viscosity of around 10,000 to 400,000 centipoise.

46. The method of claim 28 further comprising micronizing the amount of the mebendazole to a particle size of around 3.5 to 4.0 microns prior to the adding step.

47. The method of claim 28 wherein the first temperature range is 25-50° C. and the first period of time is 15-30 minutes.

48. An aqueous rosacea treatment composition for human skin comprising:
a mixture of a mebendazole solution and water;
wherein the mebendazole solution comprises mebendazole and a solvent that are heated together to a temperature in a temperature range of 60° C. to 90° C. for period of time of 5 to 20 minutes;
wherein the aqueous rosacea treatment composition comprises 0.01-0.10% mebendazole and 56% or more water;
wherein percentages are by weight of the aqueous rosacea treatment composition;
wherein the solvent comprises diethylene glycol monoethyl ether; and
wherein the aqueous rosacea treatment composition does not include any dimethyl sulfoxide and does not include any dimethylacetamide.

49. The topical aqueous mebendazole treatment composition of claim 22 wherein the topical aqueous mebendazole treatment composition comprises 80% or more water,
wherein the disease or the condition comprises any form of rosacea;
wherein the topical aqueous mebendazole treatment composition does not include any dimethyl sulfoxide and does not include any dimethylacetamide
wherein there is only one of the active ingredient.

50. The topical aqueous mebendazole treatment composition of claim 22 wherein the topical aqueous mebendazole treatment composition comprises 62% or more water and around 0.05-0.10% of the mebendazole by weight;
wherein the mebendazole and a solvent comprising diethylene glycol monoethyl ether are part of a benzimidazole solution that is heated to a temperature in a temperature range of 60° C. to 90° C. for period of time of 5 to 20 minutes;
wherein the topical aqueous mebendazole treatment composition comprises the benzimidazole solution and a carrier suitable for topical application to human skin having the disease or the condition;
wherein the topical aqueous mebendazole treatment composition only has one of the active ingredient and the active ingredient is the mebendazole; and
provides a 24 hour cumulative penetration of the mebendazole using a SKIN PAMPA test of around 3-5 μg.

51. The topical aqueous mebendazole treatment composition of claim 22 comprising 2.5-5.5% of the diethylene glycol monoethyl ether.

52. The topical aqueous mebendazole treatment composition of claim 49 comprising around 2.5-15% of the diethylene glycol monoethyl ether.

53. The topical aqueous mebendazole treatment composition of claim 22 wherein the topical aqueous mebendazole treatment composition has a viscosity of around 50,000 to 200,000 centipoise.

54. The topical composition of claim 1 further comprising 56% or more water.

55. The topical composition of claim 1 further comprising 80% or more water.

56. The topical composition of claim 9 further comprising 62% or more water; and
wherein the topical composition comprises around 0.05-0.10% of the mebendazole, the percentages by weight.

57. The topical composition of claim 56 wherein the topical composition provides a 24 hour cumulative penetration of the mebendazole using a SKIN PAMPA test of around 3-5 μg.

58. The topical composition of claim 7 wherein the topical composition comprises around 0.05-0.10% of the mebendazole by weight; and
provides a 24 hour cumulative penetration of the mebendazole using a SKIN PAMPA test of around 3-5 μg.
